(19) United States
(12) Patent Application Publication
(10) Pub. No.: US 2011/0104741 A1
(43) Pub. Date: May 5, 2011

(54) Screening Method for Inhibitors of Cancer Cell Invasion and Screening System Thereof

(75) Inventors: Da-Woon Jung, Seoul (KR); Jin Mi Kim, Seoul (KR); Zhong Min Che, Seoul (KR); Young-Tae Chang, Busan (KR)

(73) Assignee: Industry-Academic Cooperation Foundation Yonsei University, Seoul (KR)

(21) Appl. No.: 13/000,483

(22) PCT Filed: Dec. 24, 2008
(86) PCT No.: PCT/KR2008/007636

§ 371 Date: Jan. 20, 2011

(30) Foreign Application Priority Data
Jun. 23, 2008 (KR) 1020080059267
Dec. 15, 2008 (KR) 1020080127159

(51) Int. Cl. C12Q 1/02; C12M 1/34

(52) U.S. Cl. 435/29; 435/287.1

(57) ABSTRACT

This invention relates to a screening method for an inhibitor to cancer cell invasion, comprising the steps of: (a) co-culturing cancer cells and a carcinoma-associated fibroblasts (CAFs) in a multi-chamber containing a upper-chamber, a lower-chamber and a porous filter separating the upper-chamber from the lower-chamber; in which each cancer cells and CAFs is inoculated into the upper-chamber and the lower-chamber of the multi-chamber, and then a candidate is added to the upper-chamber; and (b) measuring the number of cancer cells passing the porous filter. According to the screening system and screening method using the same, the inhibitor to cancer cell invasion is able to be screened in a high-throughput manner.

### BACKGROUND OF THE INVENTION

### Field of the Invention

The present invention relates to a screening method for inhibitors of cancer cell invasion and a screening system thereof.

### BACKGROUND OF TECHNIQUE

Mature cancer tissues have a characteristics to invade into adjacent tissues from a lesion developed and proliferate, called as invasion. Invasion and metastasis of cancer cells is an indispensible relationship and the invasion of cancer cells could be considered as an early stage of metastasis. The metastasis of cancer cells is mainly composed of two processes: (a) degradation of extracellular matrix by overproduction of a protease; and (b) oriented migration of cancer cells. In addition, a theory that the metastasis of cancer cells is occurred by induction of cells in an adjacent micro-environment, not cancer cell alone, has been commonly accepted. The importance of interactions with adjacent micro-environment in tissue has been enormously emerged, massively focusing on the mechanism elucidating induction and development of differentiation, proliferation and apoptosis in epithelial carcinoma, and invasion of epithelial carcinoma into a dermal analog induced by stroma cells.

On the other hand, a fibroblast is a representative stromal cell. The experimental studies how fibroblasts influence on epithelia or epithelial carcinomas and on a novel cancer therapy targeting fibroblasts have been actively carried out. As an example, it was demonstrated that the epithelial cells rapidly acquired the characteristics of cancer cells such as excessive cell proliferation, loss of cell suicide program or tissue invasion capability where fibroblasts are adjacent to epithelial cells initiating metastasis to cancer cells in breast cancer (Sadlonova et al. 2005). It was also reported that fibroblasts derived from prostate cancer promotes proliferation of prostate cancer cells and affects differentiation of cancer cells (Orimo et al. 2005). Further, tumor cells were invaded into connective tissues only in the presence of fibroblast when oral squamous cell carcinoma (OSCC) cell line was cultured in a three-dimensional manner (Che et al, 2006).

To study cancer cell invasion, an invasion assay using a conventional transwell has been generously utilized whereas the transwell having a Matrigel-coated filter was used in the conventional method (Repesh L A. 1989). The method using normal fibroblasts isolated from normal skin was disclosed to induce cancer cell invasion (Saiki I et al. 1994). Therefore, the method to examine induction of cancer cell invasion through interactions between carcinoma-associated fibroblasts (CAF) and cancer cells has not been reported yet.

Throughout this application, various publications and patents are referred and citations are provided in parentheses. The disclosures of these publications and patents in their entities are hereby incorporated by references into this application in order to fully describe this invention and the state of the art to which this invention pertains.

### DETAILED DESCRIPTION OF THE INVENTION

The present inventors have intensive studies to develop a high-throughput screening method (HTS) for isolating a novel inhibitor to cancer cell invasion. As results, we have discovered that a highly specific inhibitor to cancer cell invasion could be screened in a high-throughput manner where a substance having not only an inhibitory activity to cancer cell invasion but also no cytotoxicity to fibroblasts is selected under conditions in which carcinoma-associated fibroblasts (CAFs) and cancer cells are co-cultured in a multi-chamber (e.g, Boyden chamber) and then a candidate is added to the culture, based on the fact that CAFs induce cancer cell invasion.

Accordingly, it is an object of the invention to provide a screening method for an inhibitor to cancer cell invasion.

It is another object of this invention to provide a screening system for inhibitors of cancer cell invasion

Other objects and advantages of the present invention will become apparent from the detailed description to follow taken in conjugation with the appended claims and drawings.

In one aspect of this invention, there is provided a screening method for an inhibitor to cancer cell invasion, comprising the steps of: (a) co-culturing cancer cells and a carcinoma-associated fibroblasts (CAFs) in a multi-chamber containing a upper-chamber, a lower-chamber and a porous filter separating the upper-chamber from the lower-chamber; in which each cancer cells and CAFs is inoculated into the upper-chamber and the lower-chamber of the multi-chamber, and then a candidate is added to the upper-chamber; and (b) measuring the number of cancer cells passing the porous filter.

The present inventors have intensive studies to develop a high-throughput screening method (HTS) for isolating a novel inhibitor to cancer cell invasion. As results, we have discovered that a highly specific inhibitor to cancer cell invasion could be screened in a high-throughput manner where a substance having not only an inhibitory activity to cancer cell invasion but also no cytotoxicity to fibroblasts is selected under the condition in which carcinoma-associated fibroblasts (CAFs) and cancer cells are co-cultured in a multi-chamber (e.g, Boyden chamber) and then a candidate is added to the culture, based on the fact that CAF induce cancer cell invasion.

As described below, the method of the present invention is explained in detail according to the step.

(a) a step co-culturing cancer cells and a carcinoma-associated fibroblasts (CAFs) in a multi-chamber containing a upper-chamber, a lower-chamber and a porous filter separating the upper-chamber from the lower-chamber; in which each cancer cells and CAFs is inoculated into the upper-chamber and the lower-chamber of the multi-chamber, and then a candidate is added to the upper-chamber.

The screening method of the inhibitor to cancer cell invasion in the present invention is based on the fact that CAFs induce cancer cell invasion where CAFs and cancer cells are co-cultured.

The screening method of the present invention is carried out according to suitable modifications of a Boyden chamber assay or a transwell assay analyzing a cell migration or invasion by a conventional chemotaxis using a multi-chamber.

The basic principle of the screening method is as follows: carcinoma-associated fibroblasts (CAFs) are incubated in the lower chamber of transwell, and cancer cells are cultured in the upper chamber of transwell, so as to induce cancer cell invasion. It is analyzed whether a candidate compound added inhibits cancer cell invasion.

The cancer cell inoculated to the upper-chamber, for example, includes stomach cancer cell, liver cancer cell, lung cancer cell, breast cancer cell, ovarian cancer cell, bronchogenic cancer, nasopharyngeal cancer cell, laryngeal cancer cell, pancreatic cancer cell, bladder cancer cell, colon cancer cell, uterine cervical cancer cell, prostate cancer cell, renal cancer cell or oral squamous cell carcinoma (OSCC) cell, but not limited to.

The term “carcinoma-associated fibroblasts (CAFs)” refers to a fibroblast located in a tissue surrounding a cancer or a malignant tumor. For example, the CAF may be feasibly separated from specimens obtained by cutting a particular malignant tumor tissue. The malignant tumor tissue as an isolation source of CAF used in the present invention, for example, includes lung cancer tissue, skin cancer tissue, stomach cancer tissue, intestinal cancer tissue, colorectal cancer tissue, pancreatic cancer tissue, liver cancer tissue, thyroid cancer tissue, uterine cancer tissue, cervical cancer tissue, ovarian cancer tissue, testicular cancer tissue, prostate cancer tissue, breast cancer tissue and oral cancer tissue, but not limited to.

In the method of this invention, the carcinoma-associated fibroblasts are cultured after inoculation into the lower-chamber of the multi-chamber, inducing cancer cell invasion of the upper-chamber.

It is one of most features of the present screening method that CAF not a chemo-attractant is used to induce cancer cell invasion different to a conventional transwell-based invasion assay.

According to a preferable embodiment, the porous filter is coated with collagen and more preferably, collagen type I.

It is another feature of the present screening method that the porous filter is coated with collagen, not Matrigel™ (a gelatin-containing protein mixture secreted from mouse tumor cells) used in a conventional transwell assay. The coating by collagen not Matrigel™ may exclude an influence of a cytokine or chemo-attractant capable of being contained in Matrigel™ on cancer cell invasion induced by CAF. Therefore, the method of this invention may permit to screen a highly specific inhibitor to cancer cell invasion

The term “candidate” refers to a substance which is expected to have cytotoxicity to cancer cells per se, not CAFs, or to inhibit secretion of, or to impede function of a cytokine or a chemokine inducing cancer cell invasion secreted from CAFs.

The candidate of the present invention includes, but not limited to, a synthetic compound, a natural compound, a low-molecular-weight compound, a nucleic acid (e.g., DNA, RNA, PNA and aptamer), a protein, a sugar and a lipid.

(b) a step measuring the number of cancer cells passing the porous filter.

The candidate inhibiting cancer cell invasion may be selected by measuring the number of cancer cells passing the porous filter.

According to a preferable embodiment, the measurement of the cell number passing the porous filter is carried out according to a method that after cells passing the porous filter are immobilized and stained, the cell number immobilized is counted. According to the method known to those skilled in the art, a method for staining a cell may be carried out. For example, the method includes a utilization of a Hoechst 33258 or crystal violet dye, but not limited to.

According to a preferable embodiment, the present invention further includes a step determining the candidate as the inhibitor to cancer cell invasion when the number of cancer cells passing the porous filter in a candidate-treated group is smaller than in that in a candidate-untreated group. Where the candidate inhibits cancer cell invasion, the number of the cancer cells passing the porous filter will be smaller in a candidate-treated group than in a candidate-untreated group.

(c) a step measuring the cytotoxicity of a candidate to CAFs inoculated into the lower-chamber.

To eliminate a candidate having non-specific cytotoxicity, the cytotoxicity of the candidate to CAFs inoculated into the lower-chamber is measured, and thus the candidate representing cytotoxicity to CAFs is not selected. The candidates representing cytotoxicity to CAFs are not a specific toxic-substance to cancer cells. By excluding these candidates, only a candidate having a specific inhibitory activity to cancer cells may be screened.

For example, the cytotoxicity of candidates to CAFs may be carried out according to a MTT assay measuring cell viability of carcinoma-associated fibroblasts.

In another aspect of this invention, there is provided a screening system for an inhibitor to cancer cell invasion, wherein each cancer cells and CAFs is inoculated into an upper-chamber and a lower-chamber in a multi-chamber containing the upper-chamber, the lower-chamber and a porous filter separating the upper-chamber from the lower-chamber.

According to a preferable embodiment, there is provided the screening system in which the porous filter is coated with collagen.

The present invention relates to a screening method for inhibitors of cancer cell invasion and a screening system thereof. According to the screening system and screening method using the same, the inhibitor to cancer cell invasion is able to be screened in a high-throughput manner.

### BRIEF DESCRIPTION OF THE DRAWINGS

FIG. 1 shows compounds having an inhibitory activity on cancer cell invasion selected from a tagged triazine compound library.

FIG. 2 schematically represents a system to screen a molecule with a low molecular weight regulating tumor-stroma interactions. For screening an invasion inhibitor, a filter with a pore size of 8 μm was coated with collagen type I (45 g/30 L). 2×10⁴ OSCC cells were inoculated into an upper well of transwell having the porous filter, and each CAF of 2×10⁴, CAF-conditioned media (CM), or CCL7 was added to a lower well of transwell having the porous filter. The cells passing the filter were immobilized and stained with 0.25% crystal violet. The cells were counted under a light microscope.

FIG. 3 is a schematic diagram representing that a compound (S06) screened according to the method of the present invention prevents invasion and proliferation of a CAF-induced oral cancer cell line (YD-10B) and reduces secretion of CCL7 by CAF. Cancer cell line (YD-10B) was inoculated into an upper well of transwell having the porous filter (pore size; 8 μm), and CAF was added to a lower well of transwell. To measure cell invasion, the cells passing a collagen-coated transwell membrane were immobilized and stained. The cells were counted under a light microscope. Cell invasion was visualized using a light microscope (FIG. 3A (i)), and cells invaded were quantitated by a cell counting (FIG. 3A (ii)). By a MTT assay, a cell viability of the oral cancer cell line (YD-10B) was measured for 48 hrs after adding the compound (S06) (FIG. 2B). 2.1×10⁴ CAF were inoculated into a well of 24-well plate and treated with S06 (1 μM or 5 μM) or DMSO (control). After incubation for 24 hrs, the supernatant was collected from CAF culture media and the concentration of CCL7 was measured by a commercially accessible ELISA kit (Error=S.D.) (FIG. 3C).

FIGS. 4-6 show graphs to test the sensitivity to cytotoxicity of the triazine compound (S06) in oral cancer cell lines (YD10B, HSC-2, HSC-3 and Ca9.22) using the MTT assay. The cell viability was measured at 24 hrs (FIG. 4), 48 hrs (FIG. 5) and 72 hrs (FIG. 6) after addition of the compound (Error=S.D.).

FIG. 7 represents a result to test the sensitivity to cytotoxicity of the triazine compound (S06) in colon cancer cell lines (HCT-116, DLD-1 and HT-29) using the MTT assay. The cell viability was measured at 24 hrs after addition of the compound (Error=S.D.).

FIG. 8 represents a result to test the sensitivity to cytotoxicity of the triazine compound (S06) in lung cancer cell lines (A549, NCI-H596 and NCI-H460) using the MTT assay. The cell viability was measured at 24 hrs after addition of the compound (Error=S.D.).

FIG. 9 represents a result to test the sensitivity to cytotoxicity of the triazine compound (S06) in breast cancer cell lines (MDA-MB-231, MDA-MB-435 and MCF-7) using the MTT assay. The cell viability was measured at 24 hrs after addition of the compound (Error=S.D.).

FIG. 10 shows graphs to test the sensitivity to cytotoxicity of the triazine compound (S06) in prostate cancer cell line (PC-3) using the MTT assay. The cell viability was measured at 24 hrs (FIG. 7A) and 48 hrs (FIG. 7B) after addition of the compound (Error=S.D.).

FIG. 11 is a histogram representing that the triazine compound (S06) has no influence on cell viability of CAF. Using the MTT assay, cell viability was measured at 24 hrs or 48 hrs after treatment with a concentration of 1, 2.5, 5, 10, 25 and 50 μM, respectively (Error=S.D.).

FIG. 12 represents a result to test the sensitivity to cytotoxicity of the triazine compound (S06) in normal epithelial cells isolated from the oral using the MTT assay (Error=S.D.).

FIG. 13 shows a histogram adding conditioned media recovered from YD-10B cell line into CAF culture in a 24-well plate in the presence or absence of the triazine compound (S06; 1 mM, 2.5 mM or 5 mM) to induce CCL7 secretion. The supernatant were collected from each medium after culturing for 24 hrs, and then the concentration of CCL7 in CAF media was measured by ELISA (Error=S.D.).

FIG. 14 represents a histogram adding conditioned media recovered from YD-10B cell line into CAF culture in a 24-well plate in the presence or absence of the triazine compound (S06; 1 mM, 2.5 mM or 5 mM) to induce CXCL7 secretion. The supernatant were collected from each medium after culturing for 24 hrs, and then the concentration of CXCL7 in CAF media was measured by ELISA (Error=S.D.).

FIG. 15 is a histogram adding conditioned media recovered from YD-10B cell line into CAF culture in a 24-well plate in the presence or absence of the triazine compound (S06; 1 mM, 2.5 mM or 5 mM) to induce IL-8 secretion. The supernatant were collected from each medium after culturing for 24 hrs, and then the concentration of IL-8 in CAF media was measured by ELISA (Error=S.D.).

### EXAMPLES

### Experimental Methods

### 1. Triazine Library Compounds

1040 compounds from a tagged triazine library were dissolved in DMSO (dimethyl sulfoxide) at a concentration of 5 mM. Tagged triazine library compounds and preparation method thereof are described in a conventional reference (Facilitated forward chemical genetics using tagged triazine library and zebrafish embryo screening, Khersonsky, S. M; Jung, D. W.; Kang, T. W.; Walsh, D. P.; Moon, H. S.; Jo, H.; Jacobson, E. M.; Shetty, V.; Neubert, T. A.; Chang, Y. T; J. Am. Chem. Soc. 2003, 125, 11804-11805). For a transwell invasion assay, an anti-invasive effect was measured by treating a candidate triazine compound with a final concentration of 5 μM.

### 2. Cell Culture

### (i) OSCC (Oral Squamous Cell Carcinoma) Cells

YD-10B cell line (Lee E J et al. Exp Mol Med 2005, 37:379-90) registered in Korean Cell Line Bank, and HSC-2, HSC-3 and Ca9.22 cell lines registered in Japanese Cell Line Bank were used as oral squamous cell carcinoma (OSCC) cells in the experiments. HSC-2, HSC-3 or Ca9.22 cell line was incubated at 37° C. incubator maintaining 5% CO₂ in DMEM:Hams-F12 (3:1) supplemented with 1% penicillin/streptomycin and 10% FBS (fetal bovine serum). YD-10B cell line was cultured in media with the above composition supplemented with cholera toxin (0.1 mg/ml, Sigma), hydrocortisone (0.4 mg/ml, Sigma), insulin (5 mg/ml, Sigma), apo-transferrin (5 mg/ml, Sigma) and 3,3′,5-triiodo-1-thyronine (2 mg/ml, Sigma) additionally.

### (ii) Colon Cancer Cells

HT-29, DLD-1 and HCT-116 cell lines were kindly provided from Yonsei Cancer Research Institute. HT-29 and DLD-1 cell lines were incubated in RPMI 1640 supplemented with 1% penicillin/streptomycin and 10% FBS. HCT-116 cell line was incubated in DMEM supplemented with 1% penicillin/streptomycin and 10% FBS.

(iii) Lung Cancer Cells

NCI-H596, NCI-H460 and A549 cell lines were kindly provided from Yonsei Cancer Research Institute. NCI-H596 and NCI-H460 cell lines were incubated in RPMI 1640 supplemented with 1% penicillin/streptomycin and 10% FBS. A549 cell line was incubated in DMEM supplemented with 1% penicillin/streptomycin and 10% FBS.

### (iv) Breast Cancer Cells

MCF-7, MDA-MB-231 and MDA-MB-435 cell lines were purchased from ATCC (American Type Culture Collection) and incubated in DMEM supplemented with 1% penicillin/streptomycin and 10% FBS.

### (v) Prostate Cancer Cells

PC-3 cell line kindly provided from Yonsei Cancer Research Institute was incubated in Hams-F12 supplemented with 1% penicillin/streptomycin and 10% FBS.

### (vi) Normal Epithelial Cells

Normal epithelial cells derived from the epithelium among normal gingival tissues of teeth pulled out for orthodontics were incubated in KGM media.

(vii) Carcinoma-Associated Fibroblasts (CAF)

The oral carcinoma tissue was washed with betadine. The tissues cut using scissors were washed with PBS. Fibroblasts were incubated in DMEM:Hams-F12 (3:1) supplemented with 1% penicillin/streptomycin and 10% FBS.

### 3. In Vitro Invasion Assay

A method to screen an inhibitor to cancer cell invasion was performed according to suitable modifications of a transwell assay. The invasion of cancer cells was measured in a 24-transwell plate (Corning Inc.). A filter with a pore diameter of 8 μm was coated with collagen type I, and CAF (carcinoma-associated fibroblasts) were seeded into a lower well beneath the porous filter at a density of 2.1×10⁴ cells/well. After culturing overnight, media were replaced with serum-free media and 2×10⁴ cancer cells diluted in serum-free media were loaded on an upper well above the filter coated with collagen. A triazine compound to be screened was added to the upper side of the filter. After culturing for 48 hrs, cells passing the porous filter were immobilized with 10% formalin and stained with 0.25% crystal violet. The number of cells was counted under a microscope.

### 4. Cell Viability Test

To measure a cell viability, MTT (3-(4,5-dimethylthiazol-2-yl)-2,5-diphenyl tetrazolium bromide) assay was carried out. The cells were seeded in 96-well plate at a density of 8×10³ cells/well. After culturing overnight, a triazine compound as a candidate to be screened was added to the plate and further incubated for 24 hrs or 48 hrs. Media were replaced with serum-free media containing MTT reagents and further incubated for 3 hrs. The culture solution was removed and DMSO was added. Formazan crystal formed was dissolved and the absorbance was measured at 540 nm.

### 5. CCL7 ELISA

The amount of CCL7 secreted into cell culture media was measured using a Human CCL7 ELISA kit (R&D Systems). CAF (carcinoma-associated fibroblasts) were seeded in 24-well plate at a density of 2.1×10⁴ cells/well and cultured overnight. Media were replaced with serum-free media of 300 μl/well and YD-10B culture solution was added to the serum-free media at a density of 200 μl/well. Simultaneously, a triazine compound as a candidate or DMSO as a control was added to the plate at an equal amount with YD-10B culture solution and further incubated for 24 hrs. The culture media were collected and analyzed by ELISA.

### 6. CXCL1/GROO-α ELISA

The amount of CXCL7 secreted into cell culture media was measured using a Human CCL7 ELISA kit (R&D Systems). The method to harvest cell culture media was carried out according to the method as same as the above-described CCL7 ELISA.

### 7. CXCL1/IL-8 ELISA

The amount of IL-8 secreted into cell culture media was measured using a Human CCL7 ELISA kit (R&D Systems). The method to harvest cell culture media was carried out according to the method as same as the above-described CCL7 ELISA.

### Results

### 1. Screening of an Invasion Inhibitor

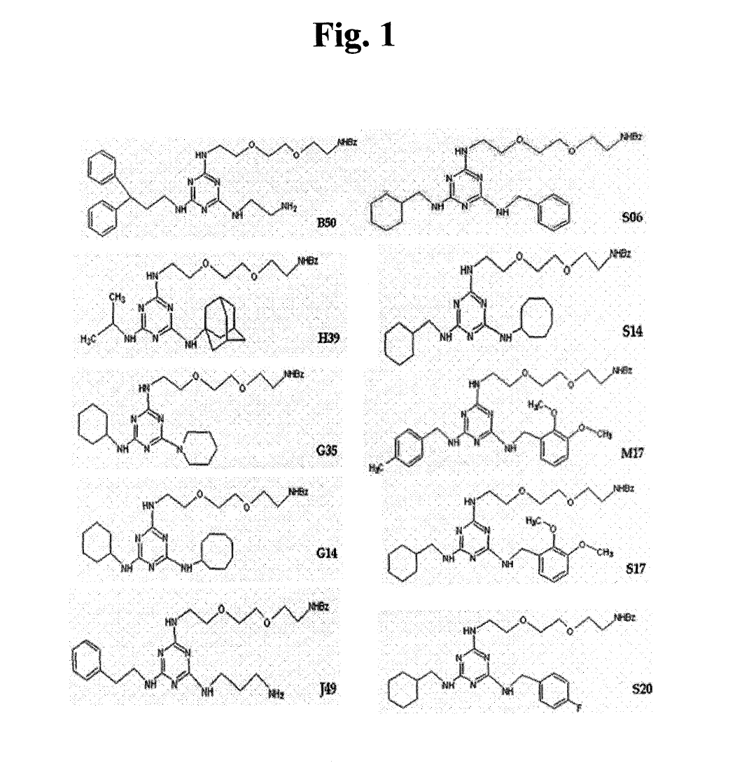

1040 compounds from a tagged triazine library were screened for anti-invasive effect. Of them, ten compounds (B50, H39, G35, G14, J49, SO6, S14, M17, S17 or S20 compound) having a strong anti-invasive effect were selected (FIG. 1).

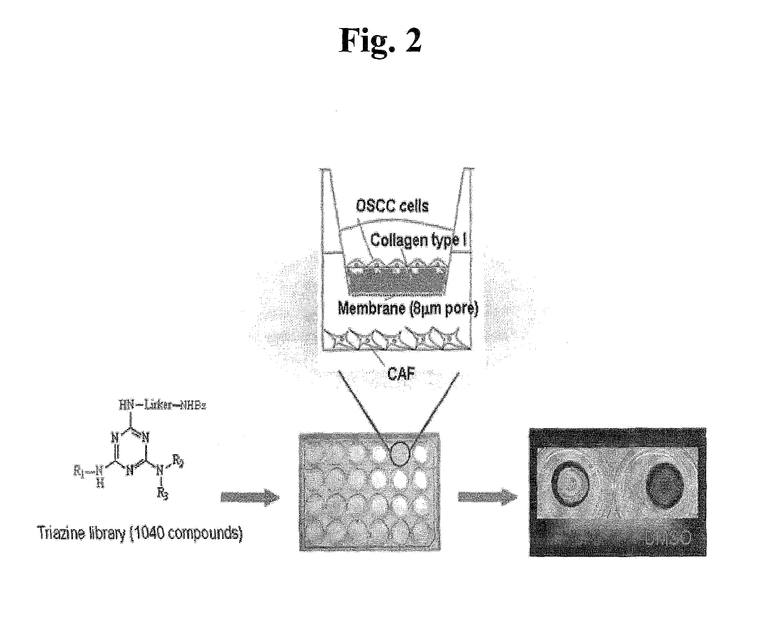

The screening method used for selection was as follows: CAF were cultured in a lower well of a transwell and OSCC cells as cancer cell were incubated in an upper well of a transwell. OSCC cell invasion was induced to investigate whether a candidate compound added inhibit cell invasion (FIG. 2). After cancer cells passing a collagen-coated filter located between the upper and lower compartment of transwell were fixed and stained, the extent to invasion of cancer cells was judged by measuring the number of stained cells. In addition, MTT assay of candidate compounds for CAF as well as measurement of the invasion-inhibitory activity described above were carried out to exclude selection of candidate compounds having an inhibitory activity caused from non-specific cytotoxicity. The compounds representing cytotoxicity to carcinoma-associated fibroblasts by a MTT assay was kept out.

Ten compounds selected as an invasion inhibitor finally were thought to have a specific cytotoxicity to cancer cells, or to prevent secretion of a cytokine or a chemokine, or to impede function of a cytokine or a chemokine inducing cancer cell invasion secreted from fibroblasts.

To confirm these possibilities, the cytotoxicity to each cancer cells, fibroblasts and normal cells, and the effect on secretion of MCP-3/CCL7 (Monocyte Chemotactic Protein-3), CXCL8/IL-8, CXCL1/GRO-α among cytokines secreted from fibroblasts were re-examined in ten compounds selected.

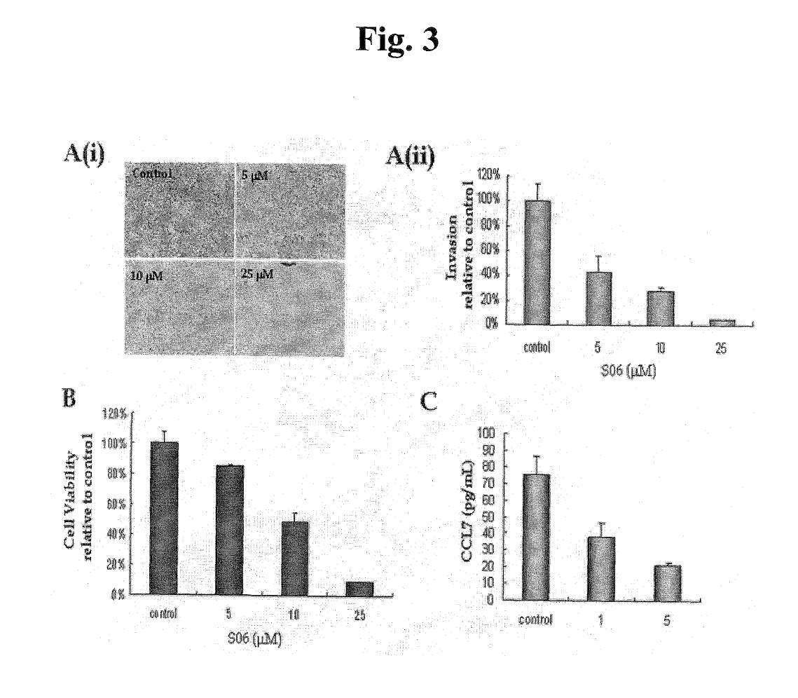

As a result, it was demonstrated that the compound S06 (the compound of formula 6) among ten compounds selected not only has a specific cytotoxicity to cancer cells but also inhibits secretion of MCP-3/CCL7 known to have a chemostaxis to cancer cells as a cytokine secreted from fibroblasts (FIG. 3).

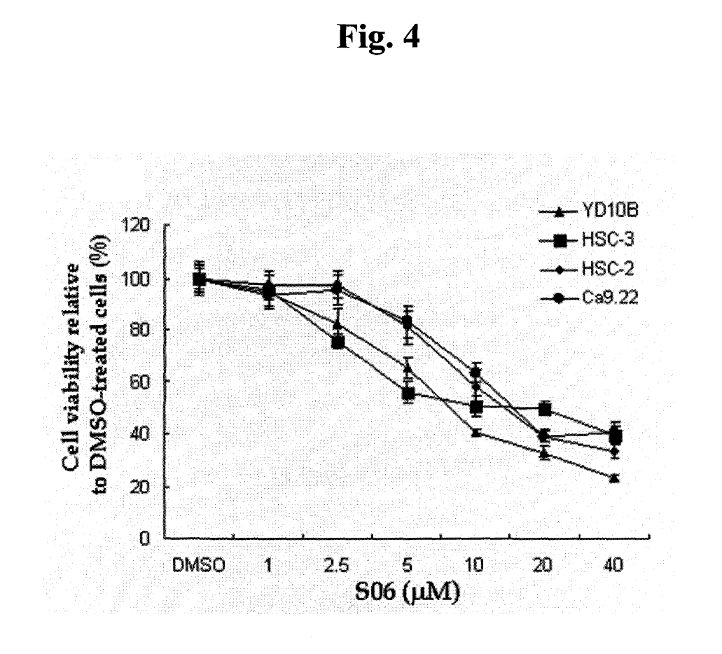

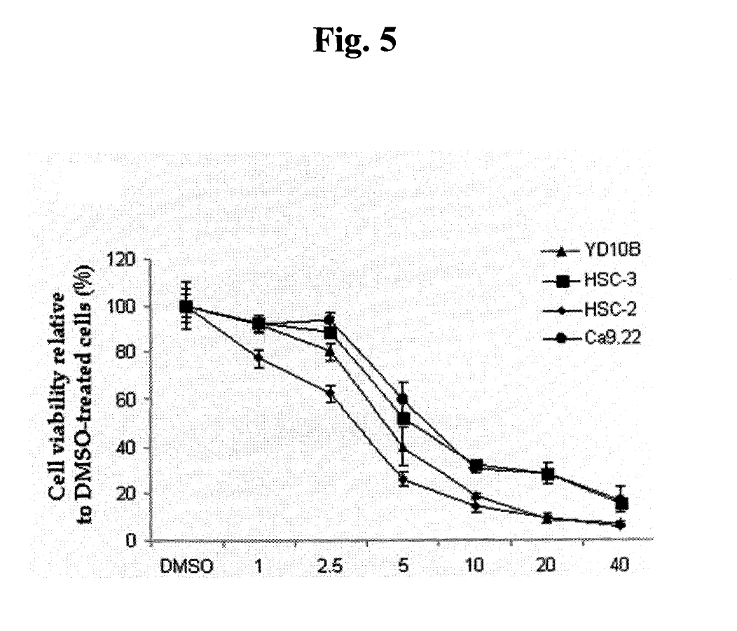

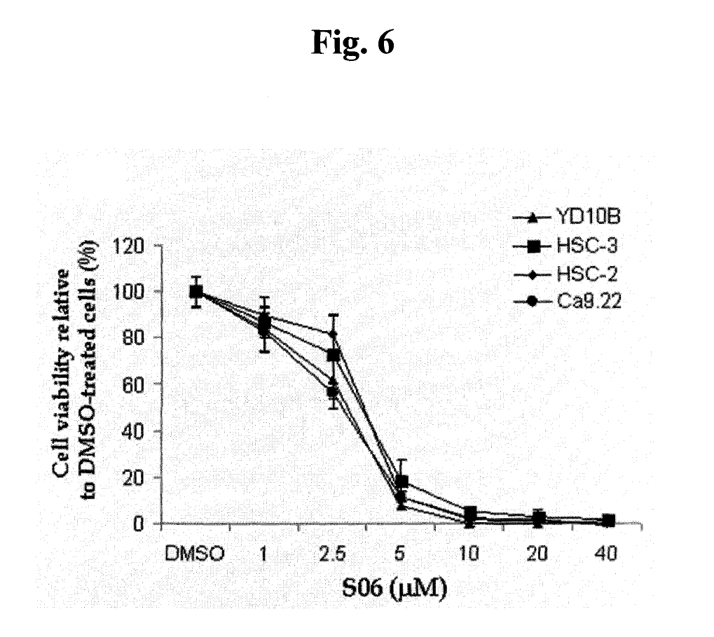

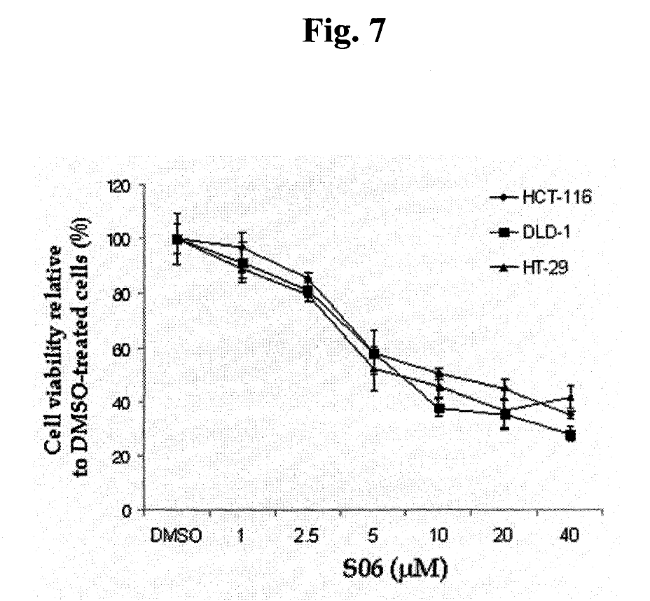

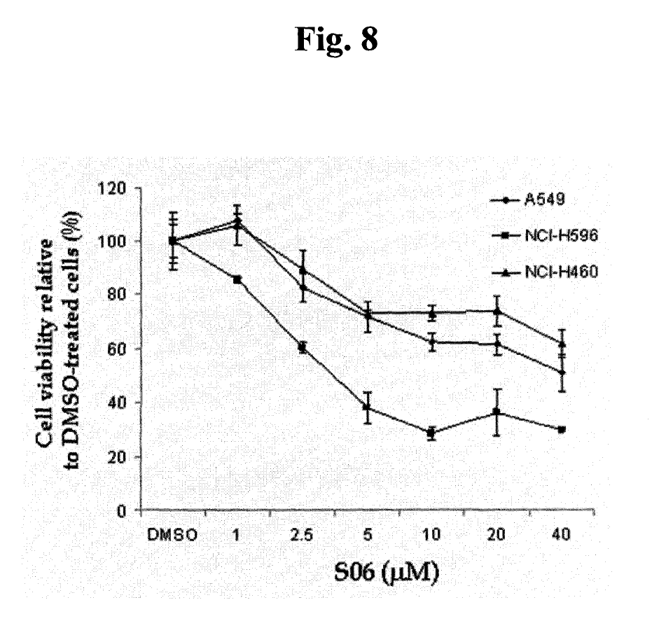

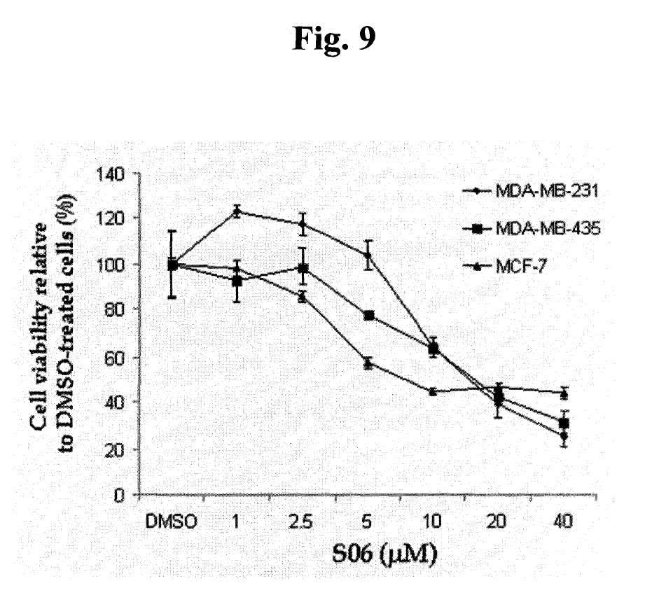

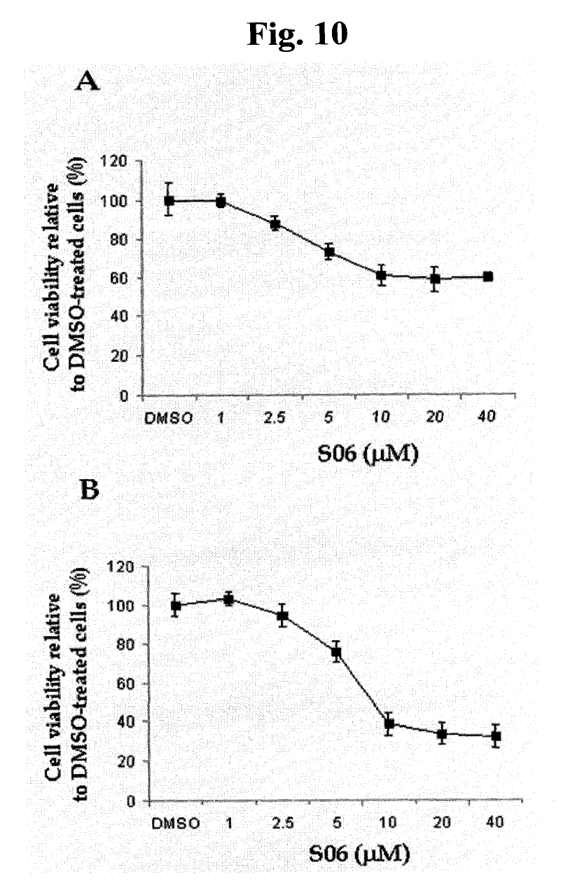

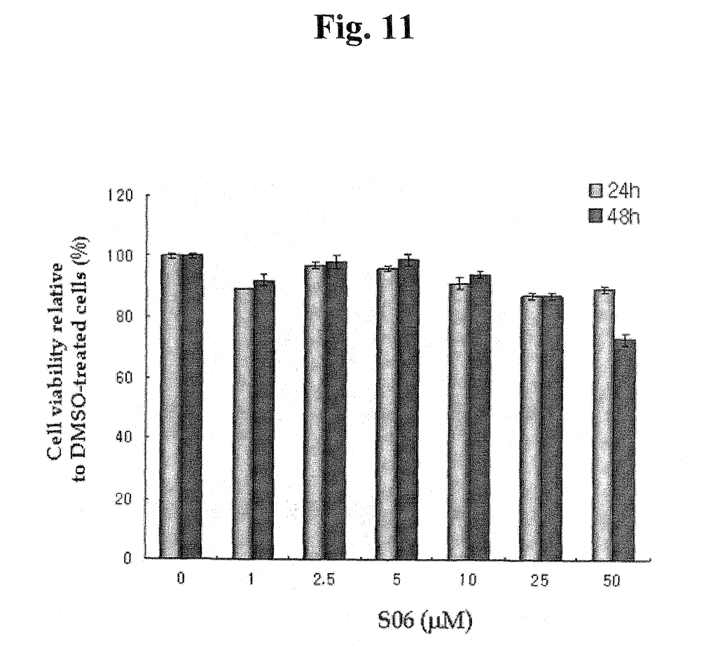

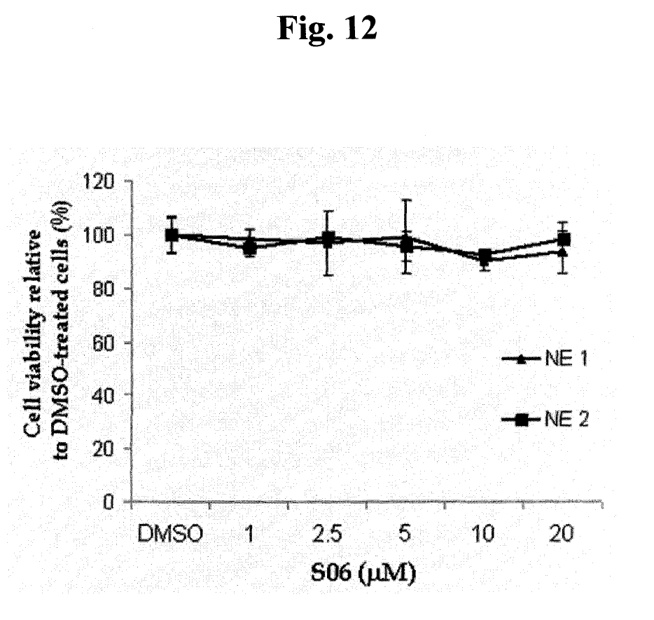

The ability of the compound S06 inhibiting a cell viability was investigated using a MTT assay, demonstrating that the cell viability of each oral cancer (FIGS. 4-6), colon cancer (FIG. 7), lung cancer (FIG. 8), breast cancer (FIG. 9) or prostate cancer (FIG. 10) cell line was decreased. However, it was demonstrated that the cell viability is sustained up to 90% by 50 μM (24 hrs) treatment in carcinoma-associated fibroblasts (FIG. 11), above 90% and above 60% by 20 μM (24 hrs) and 80 μM (24 hrs) treatment in normal epithelial cells, respectively (FIG. 12).

### 2. Effect on Cytokine Secretion of Fibroblasts by the Invasion Inhibitor

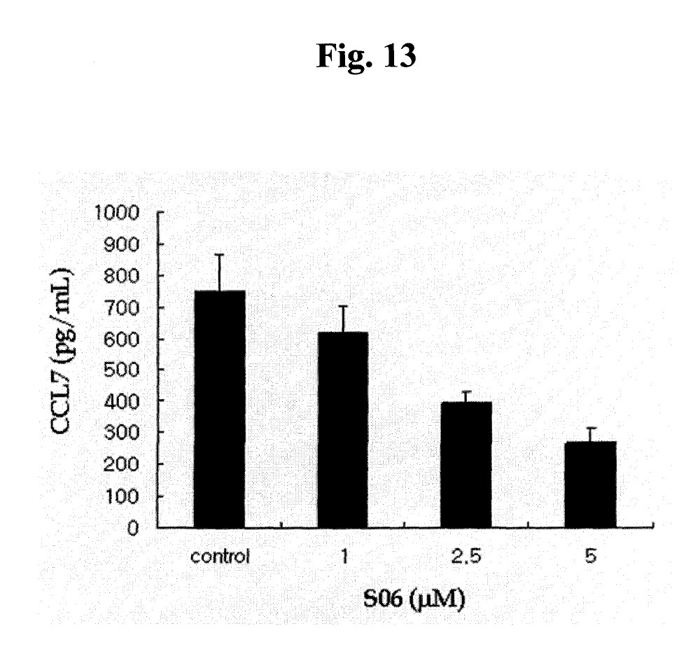

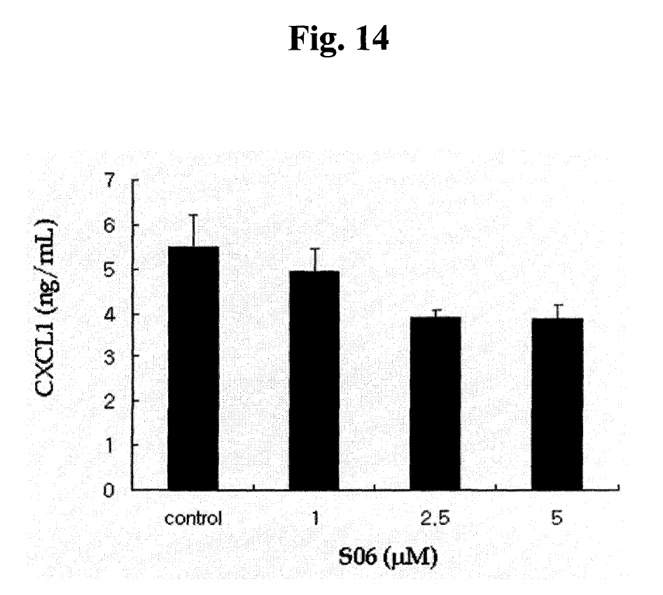

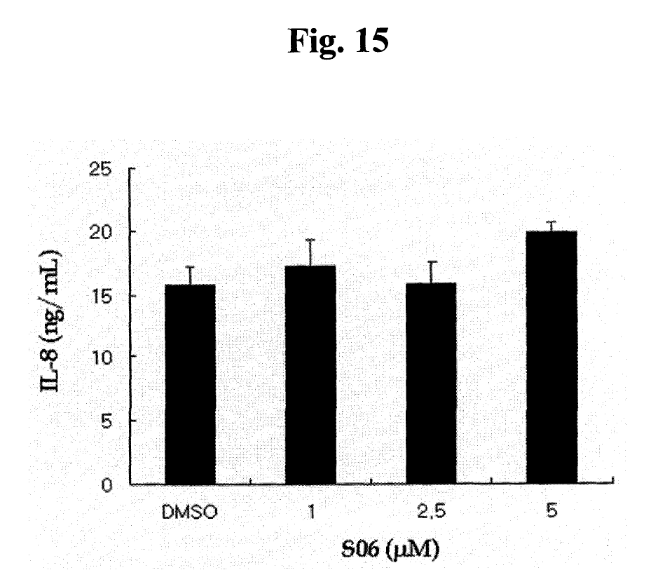

The present inventors investigated how triazine compound (S06) represented by the formula 6 has an effect on cytokine secretion in carcinoma-associated fibroblasts. Consequently, secretion of MCP-3/CCL7 and CXCL1/GRO-α increased by adding conditioned media to fibroblast culture system was decreased in a concentration-dependent manner by treating the compound S06 of formula 6 (FIG. 13 and FIG. 14) while secretion of CXCL8/IL-8 was not changed (FIG. 15).

CCL7 (Chemokine C-C motif ligand 7) is a cytokine known as a CC chemokine that was previously called monocyte-specific chemokine 3 (MCP3). CCL7 specifically attracts chemotaxis of monocytes, and regulates macrophage function. It is produced by certain tumor cell lines and by macrophages. The secretion of CCL7 was enhanced by tumor stimulation in carcinoma-associated fibroblast used in the present invention. It is thought that CCL7 secreted improves migration of cancer cells, accelerating cancer cell invasion.

CXCL1 (Chemokine C-X-C motif ligand 1) is a small cytokine belonging to the CXC chemokine family that was previously called GRO1 oncogene, Neutrophil-activating protein 3 (NAP-3) and melanoma growth stimulating activity, alpha (MSGA-α). CXCL1 is secreted by human melanoma cells, has mitogenic properties and is implicated in melanoma pathogenesis. CXCL1 is a cytokine having various activities such as angiogenesis, inflammation, wound healing, and tumorigenesis. In addition, CXCL1 is secreted by fibroblasts used in the present invention and is accelerated by cancer cell stimulation. It is thought that CXCL1 plays a function in enhancing invasion and proliferation of cancer cells.

The compounds screened in the present invention have not only a selective cytotoxicity to cancer cells but also inhibit secretion of cytokines from fibroblasts, which function to promote cancer cell invasion, providing much more remarkable treatment efficacy on cancer.

Having described a preferred embodiment of the present invention, it is to be understood that variants and modifications thereof falling within the spirit of the invention may become apparent to those skilled in this art, and the scope of this invention is to be determined by appended claims and their equivalents.

### REFERENCES

1. Sadlonova A, Novak Z, Johnson M R, et al., Breast fibroblasts modulate epithelial cell proliferation in three-dimensional in vitro co-culture. Breast Cancer Res. 2005, 7: R46-59.
2. Orimo A, Gupta P B, Sgroi D C, et al., Stromal fibroblasts present in invasive human breast carcinomas promote tumor growth and angiogenesis through elevated SDF-1/CXCL12 secretion. Cell, 2005, 121: 335-48.
3. Che Z M, Jung T H, Choi J H, et al., Collagen-based co-culture for invasive study on cancer cells-fibroblasts interaction. Biochem Biophys Res Commun 2006, 346: 268-75.
4. Saiki I, Murata J, Yoneda J, Kobayashi H, Azuma I., Influence of fibroblasts on the invasion and migration of highly or weakly metastatic B16 melanoma cells. Int J. Cancer. 1994, 56(6): 867-73.
5. Repesh L A., A new in vitro assay for quantitating tumor cell invasion. Invasion Metastasis. 1989, 9: 192-208.

What is claimed is:

1. A screening method for an inhibitor to cancer cell invasion, comprising the steps of:
(a) co-culturing cancer cells and a carcinoma-associated fibroblasts (CAFs) in a multi-chamber containing a upper-chamber, a lower-chamber and a porous filter separating the upper-chamber from the lower-chamber; in which each cancer cells and CAFs is inoculated into the upper-chamber and the lower-chamber of the multi-chamber, and then a candidate is added to the upper-chamber; and
(b) measuring the number of cancer cells passing the porous filter.

2. The method according to claim 1, further comprising, after the step (b), the step of (c) measuring cytotoxicity of the candidate to the carcinoma-associated fibroblasts inoculated into the lower-chamber.

3. The method according to claim 1, further comprising, after the step (b), the step of determining the candidate as the inhibitor to cancer cell invasion when the number of cancer cells passing the porous filter in a candidate-treated group is smaller than in that in a candidate-untreated group.

4. The method according to claim 1, wherein the porous filter is coated with collagen.

5. The method according to claim 1, wherein the cancer cell inoculated to the upper-chamber is a cancer cell selected from the group consisting of stomach cancer cell, liver cancer cell, lung cancer cell, breast cancer cell, ovarian cancer cell, bronchogenic cancer, nasopharyngeal cancer cell, laryngeal cancer cell, pancreatic cancer cell, bladder cancer cell, colon cancer cell, uterine cervical cancer cell, prostate cancer cell, renal cancer cell and oral squamous cell carcinoma (OSCC) cell.

6. The method according to claim 1, wherein the CAF inoculated into the lower-chamber is a fibroblast derived from a tissue selected from the group consisting of lung cancer tissue, skin cancer tissue, stomach cancer tissue, intestinal cancer tissue, colorectal cancer tissue, pancreatic cancer tissue, liver cancer tissue, thyroid cancer tissue, uterine cancer tissue, cervical cancer tissue, ovarian cancer tissue, testicular cancer tissue, prostate cancer tissue, breast cancer tissue and oral cancer tissue.

7. The method according to claim 1, wherein the step (b) is performed by fixing and staining cancer cells located under the filter and then measuring the number of stained cells.

8. A screening system for an inhibitor to cancer cell invasion, wherein each cancer cells and CAFs is inoculated into an upper-chamber and a lower-chamber in a multi-chamber containing the upper-chamber, the lower-chamber and a porous filter separating the upper-chamber from the lower-chamber.

9. The screening system according to claim 8, wherein the porous filter is coated with collagen.
